(19) United States
(12) Patent Application Publication
(10) Pub. No.: US 2026/0167912 A1
(43) Pub. Date: Jun. 18, 2026

(54) MULTIWELL CELL CULTURE SAMPLE PLATES WITH REDUCED DEPTH

(71) Applicant: CORNING INCORPORATED, Corning, NY (US)

(72) Inventors: Feng Li, Shrewsbury, MA (US); Gregory Roger Martin, Acton, ME (US); Sweta Nimesh Parikh, Salem, NH (US); Hilary Anne Sherman, Madbury, NH (US)

(21) Appl. No.: 19/126,859

(22) PCT Filed: Nov. 9, 2023
(86) PCT No.: PCT/US2023/037047

§ 371 Date: May 2, 2025

Related U.S. Application Data
(60) Provisional application No. 63/427,483, filed on Nov. 23, 2022.

(51) Int. Cl. C12M 1/32; C12M 1/00

(52) U.S. Cl. CPC C12M 23/12; C12M 23/38

(57) ABSTRACT

A multiwell plates for cell culture growth are disclosed having reduced maximum depths. The multiwell plates may further comprise a reduced skirt height.

### CROSS REFERENCE TO RELATED APPLICATION

This application claims the benefit of priority under 35 U.S.C. § 119 of U.S. Provisional Application Ser. No. 63/428,483 filed on Nov. 23, 2022, the content of which is relied upon and incorporated herein by reference in its entirety.

### FIELD

The present disclosure relates to multiwell cell culture sample plates, and in particular, cell culture sample plates having reduced sample well depths.

### BACKGROUND

It is known to culture cells in a multiwell culture sample plate comprising a plurality of samples wells. Such multiwell plates are typically larger than necessary such that the volume of each sample well is significantly greater than the volume of aliquot to be deposited therein. The subsequent height of the multiwell culture plate, and the samples wells thereof, may impede efficient traverse of pipettes and other instruments accessing in the sample wells. Additionally, the large volume of the sample wells adds to manufacturing cost by requiring more raw materials than necessary.

### SUMMARY

In a first aspect, a multiwell plate is disclosed, comprising a skirt comprising a bottom edge and an upper edge extending around a periphery of the skirt and a plurality of sample wells positioned within a boundary of the skirt, each sample well comprising a side wall, a bottom wall, and a maximum depth defined between an upper edge of the sample well and an inside surface of the bottom wall. The sample wells are supported by supporting structure attached to and extending between the side walls of adjacent sample wells and connecting at least one sample well of the plurality of sample wells to the skirt. A distance between the upper edge of each sample well and a plane defined by the upper edge of the skirt may be equal to or less than about 5 mm and the maximum depth of the sample wells is less than about 1.8 cm.

In a second aspect, the maximum depth of each sample well of the first aspect may be is equal to or less than about 1.5 cm;

In a third aspect, the maximum depth of each sample well of the first aspect may be equal to or less than about 1.2 cm;

In a fourth aspect, the maximum depth of each sample well of the first aspect may be in a range from about 1.0 cm to about 1.8 cm;

In a fifth aspect, a height of the skirt of any one of the first aspect through the fourth aspect and defined between the bottom edge of the skirt and an upper edge of the skirt may be less than about 2 cm;

In a sixth aspect, the height of the skirt of the fifth aspect may be less than about 1.8 cm;

In a seventh aspect, at least a portion of each sample well of any one of the first aspect through the sixth aspect may extend above the supporting structure;

In an eighth aspect, the supporting structure of any one of the first aspect through the seventh aspect may comprise a plurality of webs; and

In a ninth aspect, the supporting structure of any one of the first aspect through the seventh aspect may comprise an upper support plate encircling and joined to each sample well and further joined to the skirt.

Both the foregoing general description and the following detailed description present embodiments intended to provide an overview or framework for understanding the nature and character of the embodiments disclosed herein. The accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

FIG. 1 is a top view of an exemplary multiwell plate comprising a plurality of sample wells arranged in a rectangular array and supported by a plurality of webs, and a cover therefor;

FIG. 2 is an elevational cross-sectional view of the multiwell plate of FIG. 1;

FIG. 3 is an elevational cross-sectional view of the multiwell plate of FIG. 2, wherein the bottom wall of each sample well is spaced from a lower plate;

FIG. 4 is an elevational cross-sectional view of the multiwell plate of FIG. 2, wherein a lower plate of the multiwell plate forms the bottom wall of the multiwell plate;

FIG. 5 is a top view of an exemplary multiwell plate comprising a plurality of sample wells arranged in a rectangular array and supported by an upper plate that encircles and is joined to the plurality of sample wells;

FIG. 6 is an elevational cross-sectional view of the multiwell plate of FIG. 5;

FIG. 7 is an elevational cross-sectional view of another exemplary multiwell plate comprising a plurality of sample wells having a reduced maximum depth; and

FIG. 8 is an elevational cross-sectional view of still another exemplary multiwell plate comprising a plurality of sample wells having a reduce maximum depth and a skirt with a reduced height.

### DETAILED DESCRIPTION

Reference will now be made in detail to embodiments of the present disclosure, examples of which are illustrated in the accompanying drawings. Whenever possible, the same reference numerals will be used throughout the drawings to refer to the same or like parts. However, this disclosure can be embodied in many different forms and should not be construed as limited to the embodiments set forth herein.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art.

Ranges can be expressed herein as from “about” one particular value, and/or to “about” another particular value. When such a range is expressed, another embodiment includes from the one particular value to the other particular value. Similarly, when values are expressed as approximations by use of the antecedent “about,” it will be understood that the particular value forms another embodiment. It will be further understood that the endpoints of each of the ranges are significant both in relation to the other endpoint, and independently of the other endpoint.

Directional terms as used herein—for example, up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic with respect to arrangement of steps, operational flow, order of components, or orientation of components; plain meaning derived from grammatical organization or punctuation, and; the number or type of embodiments described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising” and “including,” and variations thereof, shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other, such as within about 5% of each other, or within about 2% of each other.

Referring to FIGS. 1-4, an exemplary multiwell plate 10 is shown. Multiwell plate 10 (e.g., microplate) comprises a frame 12 and a plurality of open-ended sample wells 14 contained within frame 12, each sample well 14 defining a volume 16 capable of receiving an aliquot of a sample to be assayed. Multiwell plate 10 may comprise a predefined number of sample wells arrayed in mutually orthogonal rows and columns. For example, multiwell plate 10 may comprise 12 sample wells, 24 sample wells, 48 sample wells, 96 sample wells, and so forth. However, the multiwell plate may have any number of sample wells 14 and is not limited to any specific number or configuration of sample wells. Each sample well 14 comprises a sample well side wall 18 and may further comprise a sample well bottom wall 20.

To prevent light transmission between adjacent sample wells 14, side walls 18 may be formed from an opaque polymeric material or filled with an inorganic TiO₂ material. For assaying techniques that require detection of very small amounts of light, as in liquid scintillation counting, pigmentation used to render frame 12 opaque may be light in color (e.g., white) so as to be highly reflective and non-absorptive to ensure high counting efficiency with respect to radioactive samples. White coloration is typically achieved with TiO₂. However, side walls 18 may be optically transparent. In some types of luminescence and fluorescence assays, side walls 18 of sample wells 14 be non-reflective and absorptive, in which case side walls 18 may be formed from a black pigmented polymer. As is commonly known and practiced, the black coloration of the polymer may be achieved by the addition of a pigment material such as carbon black to the polymer blend at concentrations readily known and practiced in the art.

Bottom wall 20 of a sample well 14 may be formed from a transparent material. The transparent material may be, for example, a polymer material, an inorganic material such as glass, e.g., boroaluminosilicate glass, pure silica, mica, or even metallic coated films. The glass material may be of a high optical quality and flatness. Optical flatness of bottom walls 20 of sample wells 14 may be desirable when the multiwell plate 10 is used for microscopic viewing of specimens and living cells within the sample wells 14. Bottom walls 20 of sample wells 14 may be formed from a glass sheet having a thickness similar to microscope slide cover slips, which are manufactured to match the optics of a particular microscope lens. Although bottom walls 20 may be of any thickness, for microscopic viewing bottom wall thickness 22 may be less than or equal to 500 micrometers and with a flatness that may exhibit a variation in a range from about zero micrometers to about 10 micrometers across the diameter of the outer bottommost surface of an individual sample well 14. However, in further embodiments, bottom wall 20 may be curved, e.g., conical. One or more chemically active coatings (not shown) may be added to an upper surface 23 of bottom walls 20.

In aspects, frame 12 may comprise a peripheral skirt 24, and a support structure supporting the plurality of sample wells 14. Skirt 24 further comprises a top edge 26 and a bottom edge 28, wherein a height 30 of skirt 24 is defined between top edge 26 and bottom edge 28. The support structure may comprise a plurality of webs 32 extending between sample well side walls 18. In addition, a subset of webs 32 may extend from peripheral sample well side walls of the array of sample wells to adjacent surfaces of frame 12 (e.g., skirt 24). Accordingly, each sample well 14 may be supported by a plurality of webs 32. Together, the plurality of webs 32 join each sample well side wall 18 to the sample well side wall of an adjacent sample well, and the sample web side walls of peripheral sample wells of the array of sample wells to frame 12 (e.g., skirt 24). The interconnectedness of frame 12, sample well side walls 18, and webs 32 joining them, provide rigidity to multiwell plate 10 and the individual sample wells 14 comprising it.

Frame 12 may further comprise a bottom portion 34. Bottom portion 34 may be joined to skirt 24 about a lower periphery of skirt 24 and a periphery of bottom portion 34, such as by an adhesive or by plastic welding (heat sealing). Although bottom portion 34 as a whole may be substantially flat, it may have relief features formed on one of its surfaces such as ridges, curves, lenses, raised sections, diffraction gratings, dimples, concentric circles, depressed regions, etc. Such features may be located on bottom portion 34 such that they shape or otherwise become features of bottom walls 20 and may in turn enhance the performance of an assay, enhance or enable detection (as in the case with lenses and gratings), or serve to mechanically facilitate bonding with frame 12 and/or sample wells 14. These relief features may be formed by any number of known methods including vacuum thermoforming, pressing, chemical etching, laser machining, abrasive machining, embossing, or precision rolling, depending on the material of bottom portion 34.

In some embodiments, skirt 24 may comprise a shoulder 36 configured to support a cover 38 for multiwell plate 10 including a rim 40 sized to fit over an upper portion of multiwell plate 10 such that a bottom edge 42 of rim 40 is supported on shoulder 36. Top edge 26 that extends around a perimeter of skirt 24 may define a first plane 44. Each sample well 14 comprises an upper edge 46 such that the resulting plurality of upper edges 46 define a second plane 48. Second plane 48 may be parallel with first plane 44. Additionally, second plane 48 may be spaced from first plane 44 by a first distance 50. Furthermore, each web 32 comprises an upper web edge 52 such that the resulting plurality of upper web edges 52 define a third plane 54. Third plane 54 may be spaced from second plane 48 by a second distance 56 and from first plane 44 by a third distance 58 that is the sum of first distance 50 and second distance 56. Third plane 54 may be parallel to second plane 48.

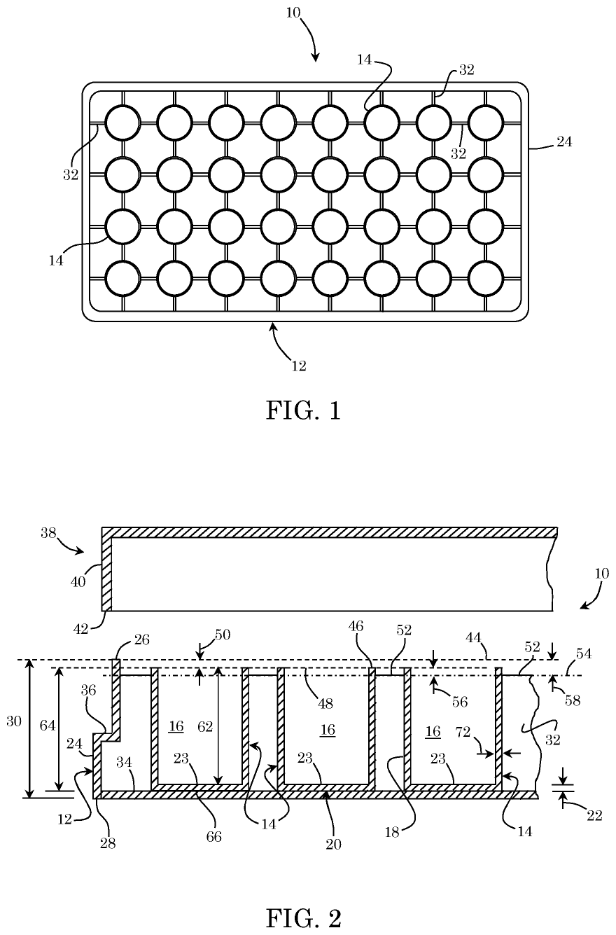

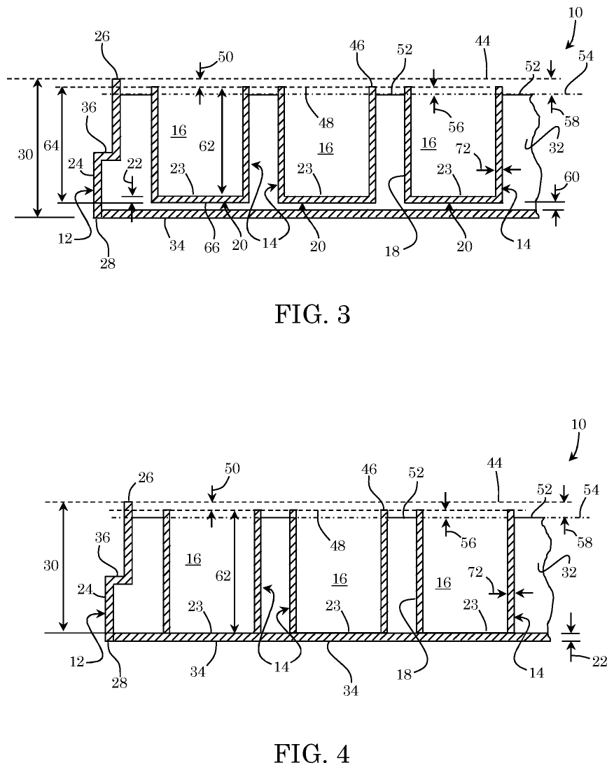

In some embodiments, bottom walls 20 of the plurality of sample wells may be in contact with bottom portion 34 of frame 12 (see FIG. 2). In other embodiments, bottom walls 20 of the plurality of sample wells may be spaced from bottom portion 34 of frame 12 by a distance 60 (see FIG. 3). In still other embodiments, bottom portion 34 of frame 12 forms the bottoms of sample wells 14 such that sample well side walls 18 are attached directly to bottom portion 34 (see FIG. 4) and bottom portion 34 forms the bottom wall of each sample well. In such embodiments, the thickness 22 of bottom wall 20 is the thickness of bottom portion 34. Each sample well 14 comprises a maximum inside depth 62 extending between the second plane 48 (coincident with upper edge 46) and bottom inside surface 23 of the sample well. Each sample well further comprises a maximum side wall height 64 defined between second plane 48 and an outside surface 66 of bottom wall 20.

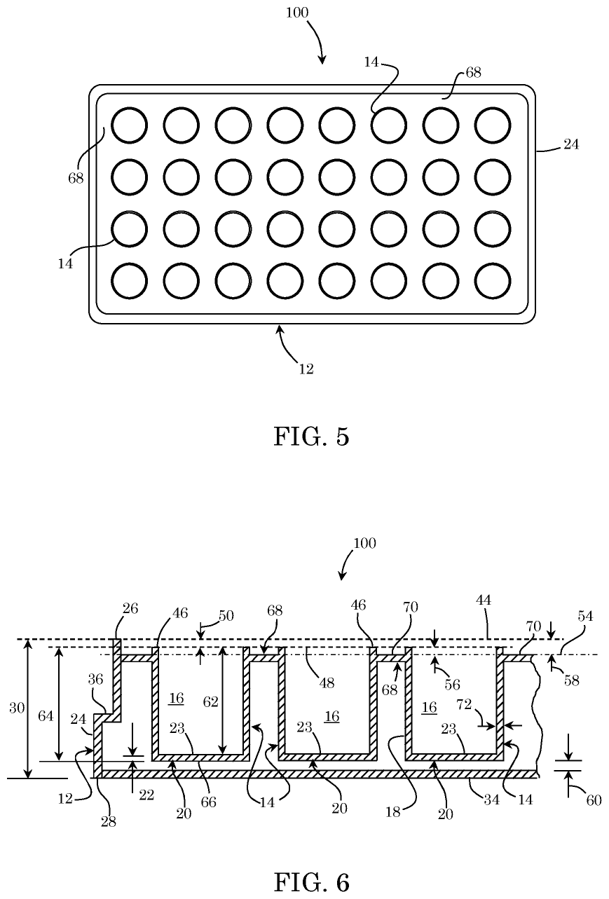

In still other embodiments, the support structure of an exemplary multiwell plate 100 is shown comprising an upper support plate 68 in place of webs 32 (see FIGS. 5 and 6), wherein upper plate 68 encircles and joins to each sample well 14 around a periphery of the sample well (e.g., sample well side wall 18). Upper support plate 68 may be further joined to frame 12 (e.g., skirt 24) about a periphery of upper plate 68. An upper surface 70 of upper support plate 68 may be recessed below top edge 26 of skirt 24 In embodiments, upper edge 46 of each sample well 14, which represents the mouth of the sample well opening, is positioned below first plane 44 defined by the top edge 26 of skirt 24. As previously described, upper edges 46 of the plurality of sample wells 14 define second plane 48. Second plane 48 may be parallel to first plane 44 and spaced apart from first plane 44 by first distance 50. First distance 50 may be in a range from about 0.1 mm to about 5 mm, although other first distances 50 are contemplated.

Sample wells 14 can be any volume and can be made in any cross-sectional shape including without limitation, square sidewalls with flat or round bottoms, conical side walls with flat or round bottoms, and/or combinations thereof. Sample well maximum depth 62 determines at least in part the volume 16 of the sample well, which is usually significantly larger than the volume needed for the cell culture medium to be added to the sample well. For example, individual sample well volume for a typical 96-well multiwell plate 10 may be about 300 microLiter (μL) per sample well, while routine cell culture operations may use up to about 100 μL per sample well of culture media. Similarly, individual sample well volume for a 24-well multiwell plate 10 may be about 3.5 milliLiter (mL) per sample well while routine cell culture operations may use about 0.5 mL to about 1.0 mL per sample well. Put another way, the typical culture medium height in a sample well of a 24-well multiwell plate may be about 0.25 centimeter (cm) to about 0.5 cm while the sample well depth is typically about 2 cm. The sample well depth needed to provide the disproportionately large sample well volume compared to the actual volume needed for the culture medium may limit the maneuverability of pipets and pipet tips of liquid handling devices (automatic or manual), especially in smaller sample well formats such as 24-sample well or 96-sample well multiwell plates. In some cell culture procedures (e.g., clone picking, dome culture for organoid culture) this can add ergonomic stress to the technician operating the liquid handling devices. Additionally, the deeper-than-necessary maximum sample well depth 60 means an unnecessary amount of polymer resin is needed for multiwell plate production, increasing the cost of manufacture.

Accordingly, a multiwell plate design having reduced sample well depth (e.g., sample well wall height) is disclosed that may be ergonomically more beneficial than current multiwell plates and may aid in cost reduction for the manufacturer by reducing raw material use.

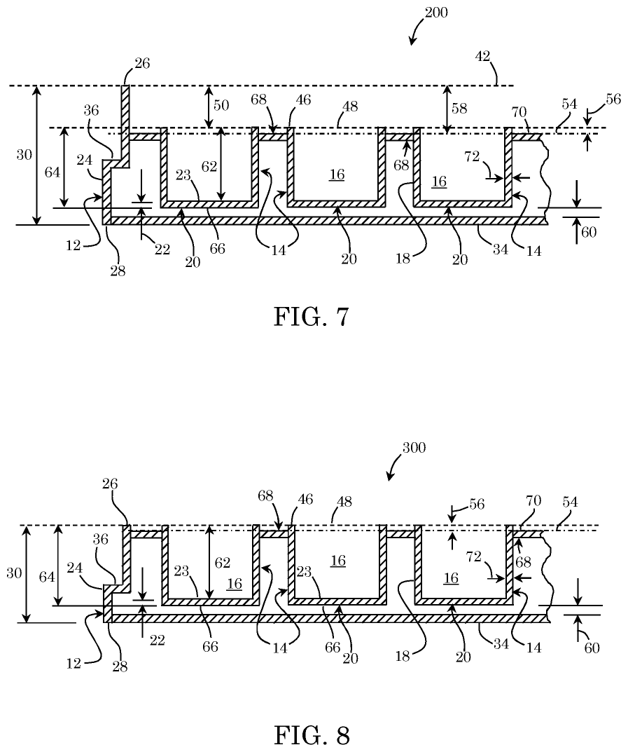

As shown in FIG. 7, a multiwell plate 200 is shown similar in design to multiwell plate 10 except that the maximum sample well depth 62 of each sample well 14 may be equal to or less than about 1.8 cm, for example equal to or less than about 1.5 cm, such as equal to or less than about 1.2 cm, or equal to or less than about 1.0 cm. Similarly, referring to FIG. 8, a multiwell plate 300 similar to the multiwell plate 100 of FIG. 7 is shown, with the exception that the height 30 of frame 12 (e.g., skirt 24) of multiwell plate 200 as well as the maximum depth 62 of the sample wells 14 may both be reduced such that the height 30 of skirt 24 may be less than about 2 cm. For example, in embodiments, the first distance 50 between first plane 44 and second plane 48 may be less about 5 mm, less than about 4 mm, less than about 3 mm, less than about 2 mm, less than about 1 mm. In some embodiments, the first distance 50 between first plane 44 and second plane 48 may be equal to zero, such that the height 30 of skirt 24 may be equal to the maximum depth 62 of sample well 14. That is, upper edge 46 of each sample well 14 may lie within second plane 48 defined by top edge 26 of skirt 24. In embodiments, the height 30 of skirt 24 may be equal to or less than about 1.8 cm, for example equal to or less than about 1.5 cm, such as equal to or less than about 1.2 cm, or equal to or less than about 1.0 cm. Similarly, the maximum depth 62 of sample wells 24 may be equal to or less than about 1.8 cm, for example equal to or less than about 1.5 cm, such as equal to or less than about 1.2 cm, or equal to or less than about 1.0 cm In embodiments, a thickness 72 of sample well side wall 18 may be in a range from about 500 micrometers to about 2 millimeters, for example in a range from about 500 micrometers to about 1 millimeter. A reduced wall thickness may provide more surface area available for cell cultures deposited in the sample wells. Additionally, thinner sample well walls may reduce mold cycle time during manufacture due to better heat removal rates, and dimensional tolerance may be improved due to reduced pin deflection during injection when forming the sample wells.

While the multiwell plates of FIGS. 7 and 8 are shown with an upper plate 68 supporting sample wells 14, multiwell plates 200 and 300 may comprise webs 32 that support sample wells 14. Additionally, any of the bottom wall configurations illustrated in FIGS. 2, 3, or 4 may be applied to the multiwell plates 200 or 300.

It will be apparent to those skilled in the art that various modifications and variations can be made to embodiments of the present disclosure without departing from the spirit and scope of the disclosure. Thus, it is intended that the present disclosure cover such modifications and variations provided they come within the scope of the appended claims and their equivalents.

1. A multiwell sample plate, comprising:
a skirt comprising a bottom edge and an upper edge extending around a periphery of the skirt;
a plurality of sample wells positioned within a boundary of the skirt, each sample well comprising a side wall, a bottom wall, and a maximum depth defined between an upper edge of the sample well and an inside surface of the bottom wall;
supporting structure attached to and extending between the side walls of adjacent sample wells and connecting at least one sample well of the plurality of sample wells to the skirt; and
wherein a distance between the upper edge of each sample well and a plane defined by the upper edge of the skirt is equal to or less than about 5 mm and the maximum depth of each sample well is less than about 1.8 cm.

2. The multiwell sample plate of claim 1, wherein the maximum depth is equal to or less than about 1.5 cm.

3. The multiwell sample plate of claim 1, wherein the maximum depth is equal to or less than about 1.2 cm.

4. The multiwell sample plate of claim 1, wherein the maximum depth is in a range from about 1.0 cm to about 1.8 cm.

5. The multiwell sample plate of claim 1, wherein a height of the skirt defined between the bottom edge of the skirt and the upper edge of the skirt is less than 2 cm.

6. The multiwell sample plate of claim 5, wherein the height of the skirt is less than about 1.8 cm.

7. The multiwell sample plate of claim 1, wherein at least a portion of each sample well extends above the supporting structure.

8. The multiwell sample plate of claim 1, wherein the supporting structure comprises a plurality of webs.

9. The multiwell sample plate of claim 1, wherein the supporting structure comprises an upper support plate encircling and joined to each sample well and further joined to the skirt.
